(19) United States
(12) Patent Application Publication
(10) Pub. No.: US 2015/0258321 A1
(43) Pub. Date: Sep. 17, 2015

(54) SEALING CONNECTOR ASSEMBLY

(71) Applicant: EDWARDS LIFESCIENCES CORPORATION, Irvine, CA (US)

(72) Inventors: Michael Higgins, Huntington Beach, CA (US); Weldon D. West, Mission Viejo, CA (US); Paul S. Van Wieren, Los Angeles, CA (US)

(73) Assignee: Edwards Lifesciences Corporation, Irvine, CA (US)

(21) Appl. No.: 14/436,262

(22) PCT Filed: Oct. 9, 2013
(86) PCT No.: PCT/US2013/064010

§ 371 Date: Apr. 16, 2015

Related U.S. Application Data
(60) Provisional application No. 61/720,116, filed on Oct. 30, 2012.

(51) Int. Cl. A61M 39/02

(52) U.S. Cl. CPC A61M 39/02; A61M 2207/00

(57) ABSTRACT

The present disclosure provides a sensor system that includes a fluid supply, one or more supply lines, a monitor, communication lines, a sensor, a catheter and a connector assembly. The connector assembly includes a housing and a tube extending out of a distal end of the housing wherein the tube is compressed against a luer connector of the catheter to form a seal between the catheter and connector assembly.

### TECHNICAL FIELD

This disclosure relates to fluidic connections for use in medical applications, and more particularly connectors between fluid lines for low flow volume applications.

### BACKGROUND

Fluidics connections on existing medical devices are usually made using standard luer lock fittings. The male fitting is either screwed into the female fitting using threads, or the male fitting may be pushed onto the female fitting. In either case, there is a static frictional force between the two fittings. For the threaded luer lock, the threads provide a mechanical advantage, forcing the male luer into the female lure. Also, the threads prevent linear translation pulling the two luers apart. The threads typically, however, do not prevent a torque from rotating the two fittings relative to each other. A problem with luer locks is that they tend to occlude when used as blood pathways and during low flow and/or low volume applications.

### SUMMARY

The present disclosure overcomes the problems of the prior art by providing a sensor system that includes a fluid supply, one or more supply lines, a monitor, communication lines, a sensor, a catheter and a connector assembly. The connector assembly includes a housing and a tube extending out of a distal end of the housing, wherein the tube is compressed against a luer connector of the catheter to form a seal between the catheter and connector assembly.

One embodiment of the present disclosure includes an access component for insertion into a proximal opening of a device configured to access the bodily fluids of a subject (hereinafter also referred to as “access device”), for example the circulatory system. The access component includes a housing and a tube. The housing comprises an internal passageway. The tube is fit within the internal passageway and extends out of the housing at a distal end thereof. The tube further comprises a fluid lumen. The tube has a distal end configured to form a compression seal with the access device when the tube and housing are fit within the proximal opening of the access device.

In one aspect, the tube has an extruded shape and may even be extruded. Also, the distal end of the tube may be configured to distort outwardly upon compression against the access device. This distortion helps to narrow the fluid lumen as it transitions to a narrower lumen of the tube. Also, it helps to fill voids at the connection between the housing and the access device.

In another embodiment, the housing is configured to friction fit within the proximal opening of the access device and to compress the tube against the access device. Also, the housing may be screwed onto the access device using luer-lock threads.

Also, the housing and tube may define aligned side openings configured to allow passage of a analyte sensor. In this manner, the fluid lumen of the tube is configured to receive and contain at least a portion of the sensor.

In another embodiment, the housing includes at least two portions configured to split apart and receive the tube. These two portions may also be configured to reattach around the tube, like a clamshell.

Another embodiment of the present disclosure includes a sensor system having a analyte sensor, at least one wire and an access component. The wire extends from the analyte sensor to an electronic device. The access component includes a housing and a tube. The housing defines an internal passageway. The tube fits within the internal passageway and extends out of the housing at its distal end. The tube defines a fluid lumen that contains the analyte sensor. Also, a distal end of the tube is configured to form a compression seal with the access device when the tube and housing are fit within the proximal opening of the access device.

In another aspect, a proximal end of the access component is configured for coupling with a flush fluid supply line. In another embodiment, the sensor system may include the flush fluid supply line. Also, the sensor system may include a pump connected to the flush fluid supply line and configured to urge flush fluid through the flush fluid supply line and the access component. The pump could also pump a calibration fluid through the access component and over the sensor.

Another embodiment of the present disclosure includes a method of making an access device. The method includes extruding a tube to define a fluid lumen. The tube is positioned in an internal passageway of a housing so that a distal end of the tube extends out of a distal end of the housing.

These and other features and advantages of the present disclosure will become more readily apparent to those skilled in the art upon consideration of the following detailed description and accompanying drawings, which describe both the preferred and alternative embodiments of the present disclosure.

### BRIEF DESCRIPTION OF THE DRAWINGS

FIG. 1 is a perspective view of a sensor system of one embodiment of the present disclosure;

FIG. 2 is a sectional view of a combined housing, tube and catheter of the sensor system of FIG. 1;

FIG. 3 is a cross-section of a housing, tube and sensor of another embodiment of the present disclosure;

FIG. 4 is a cross-section of a distal end of the tube and housing of FIG. 3 inserted into a connector of a catheter; and

FIG. 5 is a sectional view of a combined housing, tube and catheter of the sensor system of another embodiment of the present disclosure.

### DETAILED DESCRIPTION

The present disclosure now will be described more fully hereinafter with reference to specific embodiments of the disclosure. Indeed, the disclosure can be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise. The term “comprising” and variations thereof as used herein is used synonymously with the term “including” and variations thereof and are open, non-limiting terms.

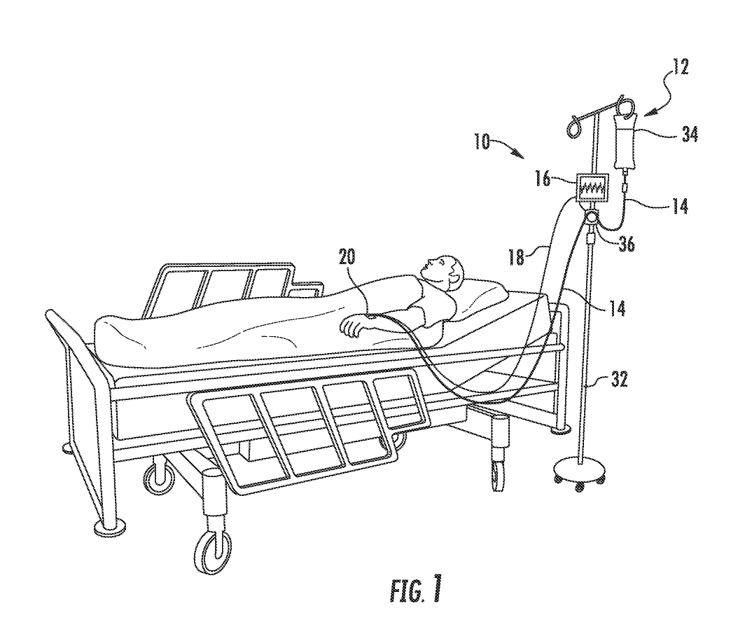

As shown in FIG. 1, embodiments of the present disclosure include a sensor system 10 that includes a fluid supply 12, one or more supply lines 14, a fluid controller 36 (e.g., a pump or pinch valve), a monitor 16, communication lines 18, a sensor assembly 20, a catheter 22 and a connector assembly 24. The connector assembly 24 includes a housing 26 and a tube 28 extending out of a distal end of the housing wherein, when inserted into the catheter 22 or other access device, the tube is compressed against a luer connector 30 of the catheter to form a seal between the catheter and connector assembly. The connector assembly can thus be a component of an over access assembly.

Referring again to FIG. 1, the fluid supply 12 in one embodiment may include some type of fluid reservoir that facilitates operation of the sensor assembly 20 and sensor system 10. For example, the fluid supply 12 may include a pole 32 supporting one or more bags 34, such as a bag of saline, or other flushing solution, and a calibration solution of an analyte, or other compound, of known concentration. Elevation of the bag 34 on the pole 32 generates head that works with the fluid controller 36 to control alternating flush, draw and calibration cycles for the sensor system 10.

It should be noted that the fluid supply 12 could also include reservoirs or existing sterile fluid supply lines or other fluid containers and need not be customized or limited to the illustrated embodiment. Generally, the fluid supply 12 is some type of liquid, gas or otherwise flowable compound that facilitates operation of the sensor system 10, such as by flushing the sensor assembly 20, connector assembly 24 and catheter 22, calibrating the sensor assembly 20 and/or administering fluids to the patient. Also, the fluid supply 12 could be configured to communicate with a pressure or other sensors combined with the sensor system 10. In yet another alternative, the sensor system 10 may not include a fluid supply 12 at all, wherein the sensor assembly 20 does not require or benefit from flushing, calibration, etc.

Regardless, in the illustrated embodiment, the supply line 14 extends down from the fluid supply 12 and includes a relatively flexible plastic tube that passes through the fluid controller 36 on its way to connect to the connector assembly 24 and the catheter 22. Preferably, the line 14 is sufficiently flexible that it can extend through and be manipulated by the fluid controller 36. As described above, however, embodiments of the sensor system 10 may not even need fluid supply and/or the fluid supply 12 may include a range of configurations. By extension, the supply line 14 may not be needed or may be modified to fit and facilitate other configurations, including the use of multiple supply lines from multiple fluid sources.

Referring again to FIG. 1, the fluid controller 36 is also supported by the pole about midway down, below the bag 34, and above the height of the catheter 22. The fluid controller 36 in the illustrated embodiment includes one or more pinch valves which compress and roll along a portion of the lines 14. By pinching and rolling, these valves meter out fluid during flushing or calibration cycles and can draw blood (or other fluid) samples by moving in reverse.

For instance, the continuous fluid column that extends down from the fluid controller 36 through the supply lines 14, the connector assembly 24 and to the catheter 22, is drawn back when the pinch valve reverses and causes a vacuum to arise in the catheter. This draws a sample of bodily fluid e.g., blood, into the catheter 22 and the connector assembly 24. In the other direction, the fluid controller 36 can either release to allow a steady drip from the bags 34 or can pinch and advance to drive flushing or calibrant solution through the connector assembly 24, the connector assembly 24 and the catheter 22.

The fluid controller 36 may include other types of pumping mechanisms that facilitate fluid movement and sample drawing into the catheter 22 and connector assembly 24. Especially useful are pumps that control low volumes and are lower-risk for use with patient fluid systems. For example, a peristaltic pump works well and, in addition to the pinch pump, maintains the sterile integrity of the tubing of the supply lines 14.

The monitor 16, as shown in FIG. 1, is mounted on the pole 32 and includes a display and some type of input-output device, such as a keyboard, buttons or a controller. The monitor 16 includes a processor, memory and other computer functions that allow it to receive and process signals from the sensor assembly 20. Also, the monitor 16 may be configured to communicate with and/or control the operation of the fluid controller 36 through the various above-described cycles.

The communication lines 18 extend from the monitor to the sensor assembly 20 coupled to the connector assembly 24. These lines communicate the signal generated by the sensor 44 and can also supply power that energizes the sensor assembly 20. It should be noted that the functions of the monitor 16 could be performed by a range of distributed computing systems that have processors, memory and other capacities at various location interconnected by network and other forms of communication. Also, the communication lines 18 could be removed in favor of wireless communication, and the sensor assembly 20 may be configured for wireless, portable communication via a network to a remotely housed computing facility with a central database.

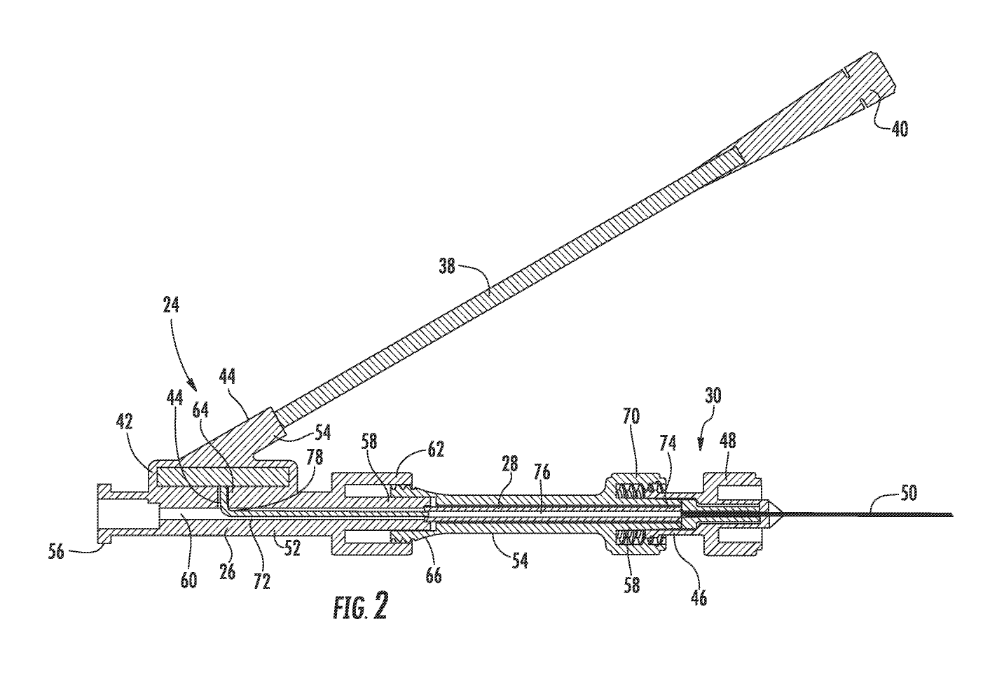

Referring now to FIG. 2, an embodiment of the disclosure including the sensor assembly 20, catheter 22 and connector assembly 24 are shown. The sensor assembly 20 includes a signal cable 38, a signal cable connector 40, a sensor puck 42 and a sensor 44. Sensor puck 42 can be constructed of a compressible, conductive material or of a compressible nonconductive material having portions thereof that are conductive, for example, to mate with electrical contacts, wires, pads, etc. The signal cable connector 40 is configured to easily mate to a congruent connector at a distal end of the communication lines 18 to establish communication with the monitor 16. The sensor signal cable 38 is configured to resiliently bend for dynamic use and intersects the housing 26 at an acute angle for lower connection strain. It attaches at its distal end to the sensor puck 42 which, in this embodiment, includes electronics in the form of a board configured to connect standard wiring with platinum wires of the sensor 44. In other embodiments, some intelligence, such as an EPROM, RF transmitter, etc. could be included in the sensor puck 42, or, no sensor puck employed at all. The sensor 44 in one embodiment is a wire sensor with a working, blank, optionally a counter electrode, and reference electrode wires that extend from the sensor puck 42 through the housing 26 and into the tube 28. In one aspect the working, blank, optional counter electrode, and reference electrode of sensor 44 are positioned near the distal tip of the wire sensor. In another aspect, the working, blank, optional counter electrode, and reference electrode of sensor 44 are positioned in proximity to sensor puck 42.

Although the connector assembly 24 of the illustrated embodiment is particularly well-suited for use with an elongate wire electrode, it should be noted that the connector assembly's ability to seal two fittings together can be employed with other sensor types, such as flexible substrate sensors, or fiber optic sensors or could even be used as a general connector. For example, the connector assembly 24 could be used to connect two fluid lines where it is desirable to eliminate dead space at the connection point. Also, although the elimination of dead space is particularly advantageous for blood lines due to avoidance of clotting and embolic events, embodiments of the connector assembly 24 may be used with other types of fluids, gasses or flowable materials.

The catheter 22 shown in FIG. 2 includes a luer receptacle 46 of the luer connector 30, a luer nut 48 and a sampling tube 50. The luer receptacle 46 has an annular shape defining an opening configured to receive a distal end of the connector assembly 24. A proximal end of the luer receptacle 46 includes threads or a flange configured to mate with the same on the connector assembly 24. The luer receptacle 46 is connected to a proximal end of the sampling tube 50. The luer nut 48 also has an annular shape and is rotatably mounted about the sampling tube 50 adjacent and distal to the luer receptacle 46. The annular shape opens distally and included on the inside of the luer nut 48 are threads allowing it to be attached to an existing catheter line in a patient, wherein the small-diameter sampling tube 50 “sleeves” into the catheter blood sampling line.

The small diameter of the sampling tube 50, for example 0.010 inch, and small amounts of blood that are drawn for operation of the sensor system 10, make for a difficult transition to standard-sized supply lines and other fluid components configured for larger volumes. For example, the system sensor 10 may be employed over a 72 hour period and sample blood with 40 to 100 L volumes in 5 to 10 minute cycles. With a 5 minute target blood glucose cycle and an approximate 90 second time window for draw volume, the maximum draw rate is about 200 mL/hour. For such volumes, the impact of even a small amount of dead space can be significant.

The small diameter sampling tube 50 can have a range of configurations to facilitate small volume sampling. In one embodiment, the sampling tube 50 has a constant 0.010 inch ID and a 0.025 inch OD so as to fit a range of standard-sized catheters. Also, the OD might be even smaller, such as 0.15 inch with a 0.010 inch ID, but the ID may be scaled down to keep bending stiffness high, such as down to 0.008 inch. The dimensions of the sampling tube need not be consistent through its entire length. For example, the ID of the sampling tube 50 could be larger closer to its proximal end to match up better with a larger upstream diameter of the tube 28. In other embodiments, the lumen of the sampling tube 50 need not be cylindrical in cross-section and could be elliptical or have intervening walls to define multiple, split lumens through which blood could be drawn simultaneously.

The connector assembly 24 includes the housing 26 and the tube 28. The housing, in the embodiment of FIG. 2, includes a main housing 52 and an adaptor 54. The main housing 52 has a cylindrical shape with a proximal end 56 and a distal end 58. Extending between the ends 56, 58 is a passageway 60. The proximal end 56 is configured to connect to a complementary luer fitting from the fluid supply line 14 and may include threads for a secure connection. The distal end 58 has a male luer shape having a central portion and a luer nut 62 extending around the central portion. The central portion is configured to extend into the adaptor 54 while the luer nut extends around the adaptor for a secure fit.

The passageway 60 is configured to receive therein the tube 28, such as in a friction fit, so that the tube is secure and can be manipulated as a single unit with the main housing 52. Defined in the side of the main housing 52 and in communication with the passageway 60 is a side opening 64. The side opening 64 is sized and configured to facilitate passage of the wire sensor 44 into the tube 28, as will be described in more detail below.

In one embodiment, the main housing 52 has additional portions that extend over the sensor puck 42 and the signal cable 38, as shown in FIG. 2. These portions may be integrally formed with the main housing 52 to additionally secure the delicate components together. In this manner, the signal cable 38, sensor puck 42 and sensor 44 are less likely to come apart from each other during use that includes attachment and detachment with movement of the patient.

In another embodiment, the main housing 52 is molded as two portions that can attach to each other in a clamshell arrangement. This enables the main housing to be easily assembled about the tube 28, signal cable 38, sensor puck 42 and wire sensor 44.

The adaptor 54 has a cylindrical shape with a proximal end 66 and a distal end 68. The proximal end 66 is configured to mate in a luer-type fit with the distal end 58 of the main housing 52 as described above. The distal end 68 includes a central portion and a luer nut 70, wherein the luer nut extends around the central portion. The central portion is configured to insert into the luer receptacle 46 of the catheter 22 and the luer nut 70 screws onto the catheter 22, thereby advancing the distal end of the housing 26 and tube 28 into the luer receptacle. Defined within the adaptor 54 is a passageway 76 that extends between the proximal end 66 and the distal end 68.

The tube 28 includes a proximal end 72 and a distal end 74 and defines a fluid lumen 78 extending therebetween. An external surface of the tube is preferably configured to conform with and tightly fit within the passageway 60 of the main housing 52 and the passageway 76 of the adaptor 54.

The internal surface of the tube 28 has a smooth surface with a low propensity to clog or clot when passing body fluids such as blood. For example, the tube 28 may be constructed of a smooth, extruded plastic, such as a polyurethane (which can be heparin coated), PTFE, FEP and PFA (and other “Teflon type” materials). Thus, the tube 28 advantageously bypasses the voids that are present within molded components configured for higher blood volume flow and/or at connections between the components, such as at the junction between the main housing 52 and the adaptor 54.

Preferably, the fluid lumen 78 has a shape and diameter configured to receive and allow passage of the wire sensor 44 but still allow sufficient clearance between the tube 28 and the wire sensor for the flush, calibration and draw cycles. The fluid lumen may have a diameter of 0.030 inch (7.1×10e⁻⁴ square inches in area) to 0.040 inch (1.26×10e⁻³ square inches) with a sensor of around 0.015 inch diameter (1.77×10e−4 square inches). Thus, the flow area is 5.33×10e⁻⁴ to 10.83×10e⁻⁴ square inches. Also, the area of the lumen in proportion to sensor area range from 400% to about 700%.

Other ranges are possible, although larger diameters have excess blood accumulation and smaller diameters are difficult to assemble.

Defined in a side of the tube 28 is an opening 80 configured to be aligned with the side opening 64 in the main housing 52. The aligned side openings facilitate passage of the wire sensor 44 from the sensor puck 42 into the fluid lumen 78.

Preferably, the tube 28 has a length sufficient for its proximal end 72 to start proximal to the side opening 64 and its distal end 74 to extend out of the passageway 76. The distal end 74 is configured to form a compression seal with the catheter 22 (or other access device) when the tube 28 and housing 26 are fit within the luer receptacle 46, of the catheter. Also, if constructed of a sufficiently compressible material, the distal end 74 of the tube 28 may distort outwardly under the compression force of the connection to help fill any voids or gaps in the transition between the housing 26 and the catheter 22. In addition, distortion may narrow the fluid lumen 78 at the transition to the smaller lumen of the sampling tube 50.

As described above, the tube 28 may be assembled or combined with the housing 26 by enclosing the tube between two or more split apart portions of the housing, such as in a clamshell arrangement. In another method, the tube 28 may be extended through the passageway 60 of the main housing 52 until the distal end 74 of the tube extends out of the main housing. The adaptor 54 is slid over the distal end 74 of the tube, leaving a small portion of the distal end extending out of the housing 26, and the adaptor is secured using the luer fitting to the main housing 52. The adaptor 54 is also attachable at the point-of-care when the housing 26 and tube 28 are attached to the catheter 22.

The method may also include aligning the side openings 64, 78 and extending the sensor 44 through the side openings 64, 78 into the fluid passageway 76 of the tube 28. Other assembly steps may also include attaching the sensor wiring to the sensor puck 42 and assembling other portions of the sensor assembly 20.

During use at the point of care, the combined housing 26 and tube 28 are advanced toward and connected to the catheter 22 by inserting the distal end 74 of the tube into the luer receptacle 46 of the catheter 22. The luer nut 70 of the adaptor 54 is screwed onto the threaded end of the luer receptacle 46, thereby compressing the distal end 74 against the terminating end of the luer receptacle.

At the proximal end of the housing 26, the supply line 14 is attached to establish communication of the fluid supply 12 through to the patient.

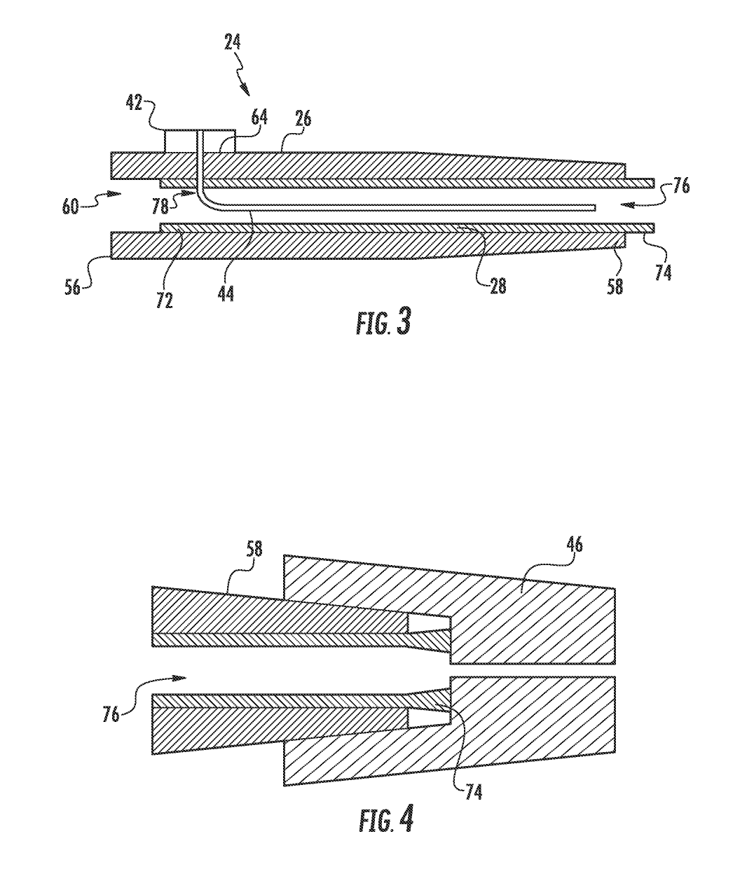

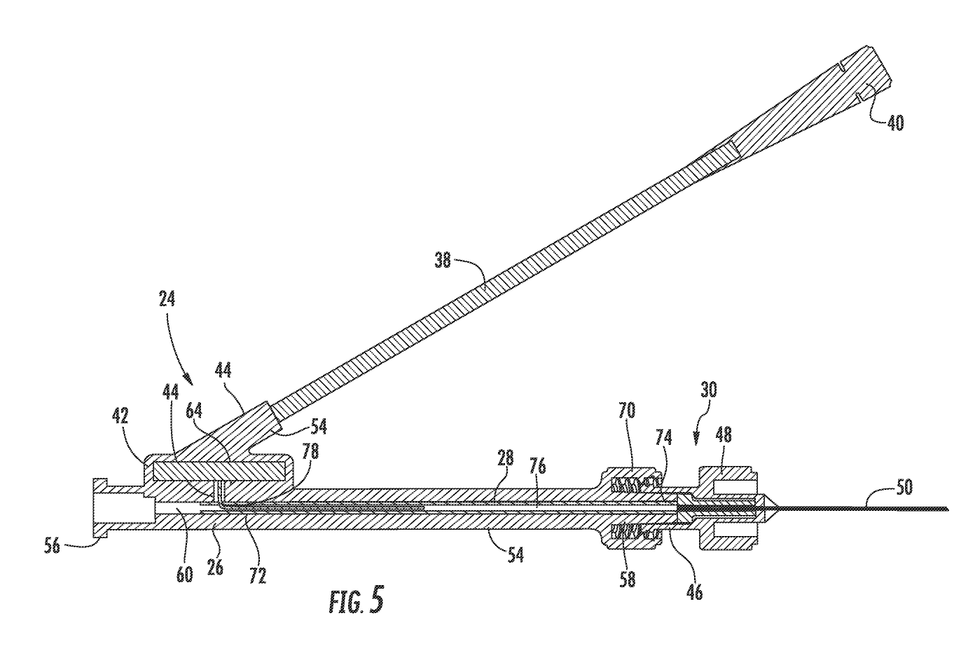

As shown in FIGS. 3 and 4, another embodiment of the present disclosure includes a housing 26 that has a tapering distal end configured to friction fit into a luer receptacle 46 of a catheter 22. As the housing 26 and catheter 22 are secured, the distal end 74 of the tube 28 is compressed in a seal against the luer receptacle 46. The walls of the tube 28 expand due to the compression, thereby narrowing the fluid passageway 76 and reducing the void space between the outer diameter of the tube and the inner diameter of the luer receptacle 46.

A shown in FIG. 5, another embodiment of the disclosure eliminates the need for intervening luer nut 62 and 66 (as shown in FIG. 2).

10 sensor system

12 fluid supply

14 supply lines

16 monitor

18 communication lines

20 sensor assembly

22 catheter

24 connector assembly

26 housing

28 tube

30 luer connector

32 pole

34 bags

36 fluid controller

38 signal cable

40 signal cable connector

42 sensor puck

44 sensor

46 luer receptacle

48 luer nut

50 sampling tube

52 main housing

54 adaptor

56 prox end of 52

58 distal end of 52

60 passageway

62 luer nut

64 side opening

66 proximal end of adaptor

68 distal end of adaptor

70 luer nut

72 proximal end of tube

74 distal end of tube

76 fluid lumen of tube

78 side opening

A number of aspects of the systems, devices and methods have been described. Nevertheless, it will be understood that various modifications may be made without departing from the spirit and scope of the disclosure. Accordingly, other aspects are within the scope of the following claims.

That which is claimed:

1. An access component for insertion into a proximal opening of an access device, the access component comprising:
a housing defining an internal passageway; and
a tube, the tube fit within the internal passageway and extending out of the housing at a distal end of the housing, said tube comprising a fluid lumen and a distal end configured to form a compression seal with the access device when the tube and housing are fit within the proximal opening of the access device.

2. An access component of claim 1, wherein the tube has an extruded shape.

3. An access component of claim 1, wherein the distal end of the tube is configured to distort outwardly upon compression against the access device.

4. An access component of claim 3, wherein a distal end of the fluid lumen narrows upon compression against the access device.

5. An access component of claim 1, wherein the housing is configured to friction fit within the proximal opening of the access device and to compress the tube against the access device.

6. An access component of claim 1, wherein the housing and tube define aligned side openings configured to receive a sensor extending therethrough.

7. An access component of claim 5, wherein the fluid lumen is configured to receive and contain at least a portion of the sensor.

8. An access component of claim 1, wherein the housing includes at least two portions that are configured to split apart and receive the tube.

9. An access component of claim 8, wherein the two portions are configured for reattachment around the tube.

10. A sensor system for connection to an access device, the sensor system comprising:
a analyte sensor;
an electronic device in communication with the analyte sensor;
an access component, the access component including:
a housing comprising an internal passageway; and
a tube fit within the internal passageway and extending out of the housing at a distal end of the housing, said tube comprising a fluid lumen containing the sensor;
wherein the tube has a distal end configured to form a compression seal with the access device when the tube and housing are fit within the proximal opening of the access device.

11. A sensor system of claim 10, wherein the tube has an extruded shape.

12. A sensor system of claim 10, wherein the distal end of the tube is configured to distort outwardly upon compression against the access device.

13. A sensor system of claim 12, wherein a distal end of the fluid lumen narrows upon compression against the access device.

14. A sensor system of claim 10, wherein the housing is configured to friction fit within the proximal opening of the access device and to compress the tube against the access device.

15. A sensor system of claim 10, wherein the tube defines a side opening and wherein the wire extends from the sensor through the side opening.

16. A sensor system of claim 15, wherein the housing also defines a side opening and wherein the wire extends from the sensor through both side openings.

17. A sensor system of claim 10, wherein the housing includes at least two portions that are configured to split apart and receive the tube.

18. A sensor system of claim 17, wherein the two portions are configured for reattachment around the tube.

19. A sensor system of claim 10, further comprising a flush fluid supply line and wherein a proximal end of the access component is configured for coupling with the flush fluid supply line.

20. A sensor system of claim 19, further comprising a pump connected to the flush fluid supply line and configured to urge flush fluid through the flush fluid supply line and the access component.

21. A sensor system of claim 20, wherein the pump is further configured to draw body fluid through the access device, into the tube and over the sensor.

22. A method of making an access device, the method comprising:
extruding a tube comprising at least one fluid lumen; and
positioning the tube in an internal passageway of a housing so that a distal end of the tube extends out of a distal end of the housing.

23. A method of claim 22, further comprising defining a side opening in the extruded tube, the side opening configured to allow passage of at least a portion of a sensor assembly therethrough.

24. A method of claim 23, further comprising defining a side opening in the housing and aligning both side openings.

25. A method of claim 24, further comprising inserting a sensor through the side openings and into the fluid lumen of the tube.

26. A method of claim 25, further comprising assembling the housing by attaching at least two portions together about the tube.
